(19) United States
(12) Patent Application Publication
(10) Pub. No.: US 2014/0356291 A1
(43) Pub. Date: Dec. 4, 2014

(54) IDH1-Mutated Human Glioblastoma Cell Lines and Xenografts

(71) Applicant: DUKE UNIVERSITY, Durham, NC (US)

(72) Inventors: Hai Yan, Chapel Hill, NC (US); Darell D. Bigner, Mebane, NC (US)

(73) Assignee: DUKE UNIVERSITY, Durham, NC (US)

(21) Appl. No.: 14/364,029

(22) PCT Filed: Dec. 10, 2012
(86) PCT No.: PCT/US12/68774

§ 371 Date: Jun. 9, 2014

(30) Foreign Application Priority Data
Dec. 8, 2011 (US) 61568398

(51) Int. Cl. C12N 5/079; C12Q 1/02; A61K 49/00; C12Q 1/68

(52) U.S. Cl. CPC C12N 5/0622; C12Q 1/68; C12Q 1/025; A61K 49/0008
USPC 424/9.2; 435/368; 435/6.1; 435/29

(57) ABSTRACT

IDH1-mutant cell lines and xenografts (e.g., IDH1R132H heterozygous and IDH1R132H homozygous) are derived from human glioblastoma multiforme (GBM) samples. Methods use said cells and xenografts as tools for determining the impact of IDH1R132H on cancer properties including cellular morphology, tumorigenesis, DNA, apoptosis, and metabolic profiles. Methods also use these cell lines for the screening and identification of candidate therapeutic targets.

This application claims the benefit of provisional application U.S. No. 61/568,398 filed Dec. 8, 2011, the contents of which are expressly incorporated herein.

This invention was made using funds from the U.S. National Institutes of Health, National Cancer Institute grant no. 1-R37-CA11898. The U.S. government retains certain rights in the invention under the terms of the grant.

### BACKGROUND OF THE INVENTION

Mutations in the isocitrate dehydrogenase genes IDH1 and IDH2 occur at an exceptionally high frequency (80%) in gliomas. The mutations also present in 23% of acute myeloid leukemia, and are rarely observed in other types of cancers. The frequency, specificity, and early timing of IDH mutations provide strong evidence for their importance in tumorigenesis, prognosis and therapeutics. However, the functional effects and significance of IDH mutations in human cancer have not been defined. Currently, a major limitation of the field is that there are no glioblastoma cell line or xenograft models which recapitulate IDH mutation-dependent tumor progression.

There is a need in the art for glioblastoma (GBM) cell lines and xenograft models.

### SUMMARY OF THE INVENTION

One aspect of the invention is an isolated human GBM xenograft identified as TB08-0714 (IDH1R132H homozygous) or as TB08-0537 (IDH1R132H heterozygous).

Another aspect of the invention is an isolated human GBM cell line identified as TB08-0714 (IDH1R132H homozygous) or as TB08-0537 (IDH1R132H heterozygous).

According to another aspect of the invention a method determines the effect of a test compound on DNA accumulation in an IDH1 gene-defective cell line or xenograft of TB08-0714 (IDH1R132H homozygous) or TB08-0537 (IDH1R132H heterozygous). The IDH1 gene-defective cell line or xenograft is incubated in the presence and absence of the test compound. The DNA content in the IDH1 gene-defective cell or xenograft is determined. The test compound is selected if it causes DNA accumulation in the IDH1 gene-defective cell or xenograft.

According to another aspect of the invention a method screens for potential anti-tumor agents using an IDH1 gene-defective cell line or xenograft. An IDH1 gene-defective cell or xenograft (TB08-0714 (IDH1R132H homozygous) or TB08-0537 (IDH1R132H heterozygous)) is incubated in the presence and absence of the anti-tumor agents. Cell viability or apoptosis of the IDH1 gene-defective cell or xenograft is determined. Anti-tumor agents which reduce cell viability or induce apoptosis in the IDH1 gene-defective cell or xenograft are selected.

According to another aspect of the invention a method screens for the effect of a test compound. A xenograft derived from IDH1 gene-defective cells (TB08-0714 (IDH1R132H homozygous) or TB08-0537 (IDH1R132H heterozygous) is incubated in the presence and absence of the test compound. Tumor morphology of the cells or xenograft is determined. A test compound that reduces the cancerous morphology or increases the normal morphology is selected.

These and other embodiments provide the art with cell lines which can be used for screening potential anti-tumor agents.

### BRIEF DESCRIPTION OF THE DRAWINGS

FIG. 1 provides a Kaplan-Meir curve for IDH1 mutant xenoline NOD SCID IC. The 08-0537 or 08-0714 cells were injected intracranially for this survival study. The 50% survival time is 52 days and 58 days for 08-0537 and 08-0714 respectively.

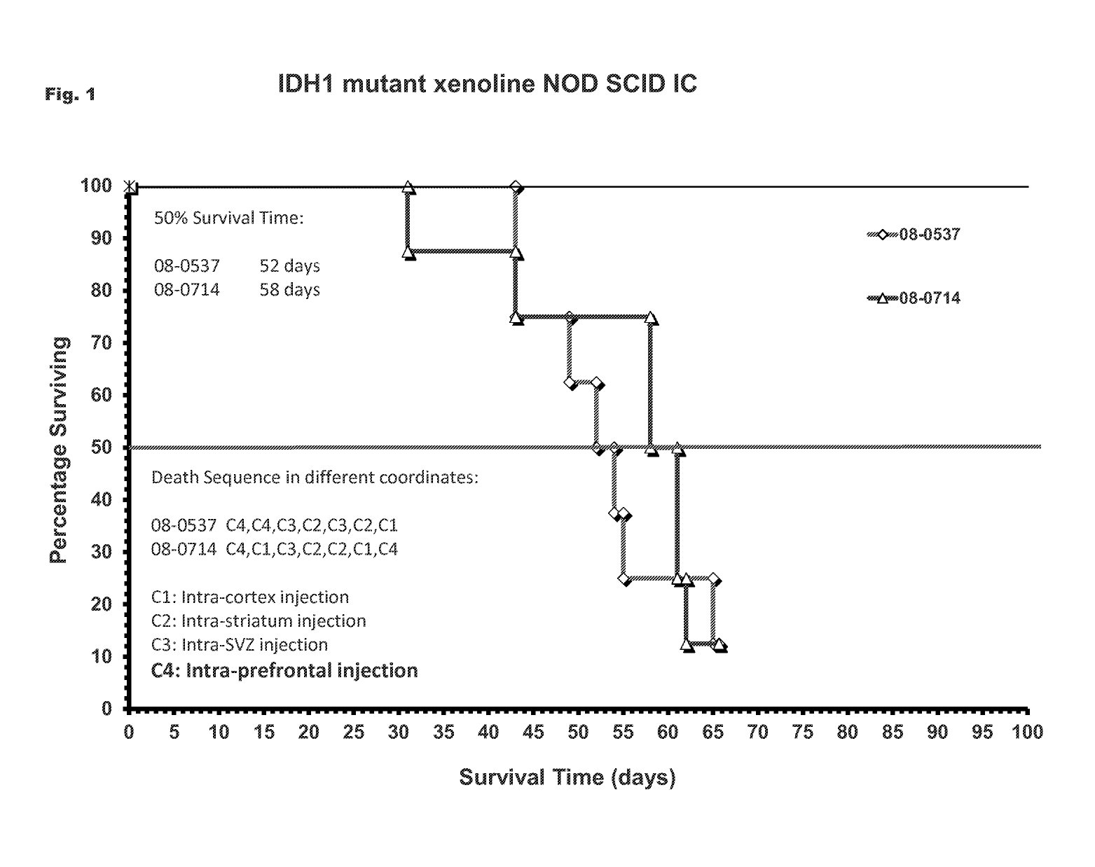

FIG. 2 shows IDH1 mutant tumor in NOD SCID mice (Xeno 08-0537, pre-frontal injection) The xenograft images were taken from NOD SCID mice that have been intracranially injected with either 08-0537 cells. The mice were sacrificed and the brains were fixed for sections and pathological assessment when the mice showed symptoms.

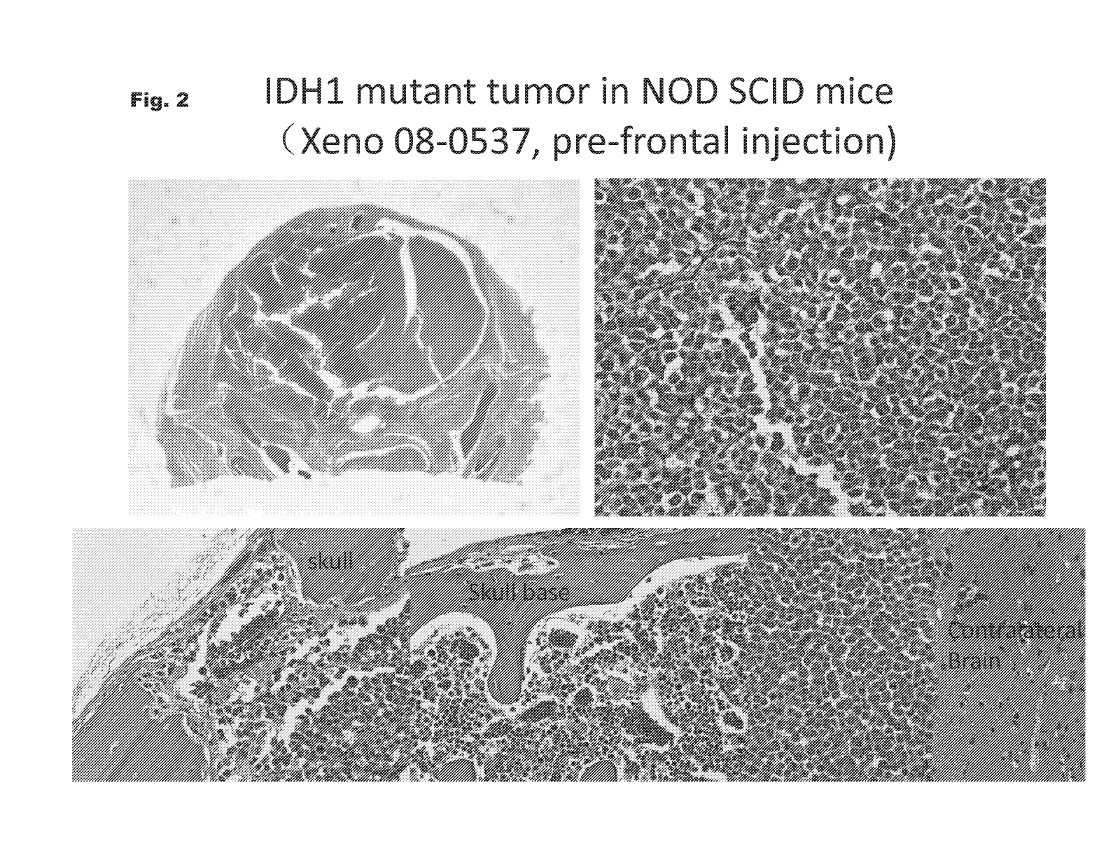

FIG. 3 shows IDH1 mutant tumor in NOD SCID mice (Xeno 08-0714, pre-frontal injection) The xenograft images were taken from NOD SCID mice that have been intracranially injected with either 08-0714 cells. The mice were sacrificed and the brains were fixed for sections and pathological assessment when the mice showed symptoms

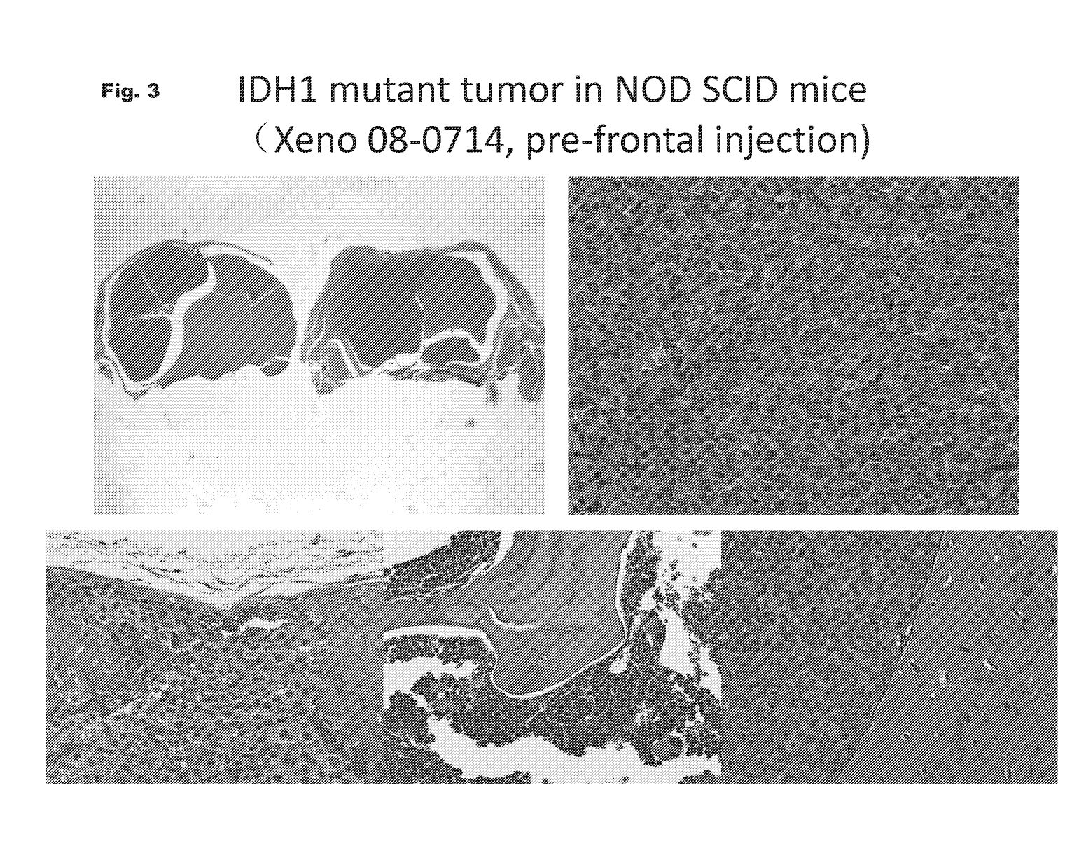

### DETAILED DESCRIPTION

IDH1 gene mutations that affect amino acid 132 of IDH1 occur in an exceptionally high frequency (more than 70%) in gliomas. IDH1 and IDH2 mutations are also present in 23% of acute myeloid leukemia, but are rarely observed in other types of cancers, suggesting that IDH1 mutants are dependent on specific cell type or cell environment. By far, the most common mutation seen in glioma patients is IDH1 R132H. The inventors have generated and isolated xenografts and cell lines having a different IDH1 status (i.e., IDH1R132H heterozygous and IDH1R132H homozygous) from dissociated glioblastoma multiforme (GBM) samples. These cell lines and xenografts are useful as tools for determining the impact of IDH1R132H on cellular biology, tumorigenesis, viability, apoptosis, and metabolic profiles. These cell lines and xenografts can also be used for the discovery of new therapeutic targets and for the screening of novel molecular therapeutic agents.

The inventors have established and characterized stable, long-term human glioblastoma cell lines and xenografts that are homozygous or heterozygous for mutations in the IDH1 gene. The established cell lines provide regents for studying tumor growth and the biological effects of IDH1. The cell lines and xenografts are useful tools to test different therapeutic approaches, e.g., chemotherapeutic, radiological, nutritional, alternative, or biological, in a relevant disease model. The cell lines can be used for studies in cell culture or can be transplanted into a laboratory animal, such as a nude mouse, to form a xenograft. Xenografts can be tested in vivo in the animal, or they can be removed and tested in vitro.

In order to study directly the effect of IDH1 gene mutations in human cancer cells the inventor(s) have generated cell lines having various IDH1 status (e.g., wild-type, IDH1R132H heterozygous, and IDH1R132H homozygous) from dissociated primary human glioblastoma samples. These cell lines can be used as a tool for determining the impact of IDH1R132H on cellular biology, tumorigenesis, and metabolic profiles. Moreover, these cell lines can be used for the testing of therapeutic targets and for the screening of molecular therapeutic agents.

Any means known in the art to generate a cell line which comprises a defective IDH1 gene can be used to obtain the IDH1 gene-defective cells. For example, an glioblastoma cell line can be used to give rise to an isogenic IDH1 negative cell line by promoterless homologous recombination (see, e.g., Waldman, T. et al. (1995) Cancer Res. 55:5187-5190, the contents of which are expressly incorporated by reference). Alternatively, as described below, a primary glioblastoma cell can be used which already has an IDH1R132H mutation. A cell with two wild-type alleles of the IDH1 gene is a gene-normal cell, for purposes of the present disclosure. A cell with one or two mutant IDH1 alleles is termed an IDH1 gene-defective cell. Preferably, the IDH1 gene-normal cell used in the assay is the same type of cell (i.e., organ source) as the IDH1 gene-defective cell. More preferably, the two cell lines are isogenic or as closely isogenic as possible.

Any of the IDH1 alleles may be engineered to be present in a cell with any of the other alleles. Any of the alleles may be present in the heterozygous, homozygous, or hemizygous state. The presence of combinations of different alleles in a single cell may modify the phenotype.

Viability and cell death can be used as ways of assessing the effects of test agents on cells. Preferably a differential effect will be observed on an IDH1 gene-normal cell and an IDH1 gene-defective cell. Any assay for such effects can be used. In whole animals, regression of tumors can be observed. Alternatively, disease-free progression can be observed. Optionally, a change in survival can be observed. It is well known in the art that viability of a cell in culture can be determined by contacting the cell with a dye and viewing it under a microscope. Viable cells can be observed to have an intact membrane and do not stain, whereas dying or dead cells having “leaky” membranes do stain. Incorporation of the dye by the cell indicates the death of the cell. The most common dye used in the art for this purpose is trypan blue. Viability of cells can also be determined by detecting DNA synthesis. Cells can be cultured in cell medium with labeled nucleotides, e.g., ³H-Thymidine. The uptake or incorporation of the labeled nucleotides indicates DNA synthesis. In addition, colonies formed by cells cultured in medium indicate cell growth and is another way to test viability of the cells.

Apoptosis is a specific mode of cell death recognized by a characteristic pattern of morphological, biochemical, and molecular changes. Cells going through apoptosis appear shrunken, and rounded; they also can be observed to become detached from culture dish. The morphological changes involve a characteristic pattern of condensation of chromatin and cytoplasm which can be readily identified by microscopy. When stained with a DNA-binding dye, e.g., H33258, apoptotic cells display classic condensed and punctate nuclei instead of homogeneous and round nuclei.

A hallmark of apoptosis is endonucleolysis, a molecular change in which nuclear DNA is initially degraded at the linker sections of nucleosomes to give rise to fragments equivalent to single and multiple nucleosomes. When these DNA fragments are subjected to gel electrophoresis, they reveal a series of DNA bands which are positioned approximately equally distant from each other on the gel. The size difference between the two bands next to each other is about the length of one nucleosome, i.e., 120 base pair. This characteristic display of the DNA bands is called a DNA ladder and it indicates apoptosis of the cell. Apoptotic cells can be identified by flow cytometric methods based on measurement of cellular DNA content, increased sensitivity of DNA to denaturation, or altered light scattering properties. These methods are well known in the art and are within the scope of the present disclosure.

Abnormal DNA breaks are also characteristic of apoptosis and can be detected by any means known in the art. In one preferred embodiment, DNA breaks are labeled with biotinylated dUTP (b-dUTP). Cells are fixed and incubated in the presence of biotinylated dUTP with either exogenous terminal transferase (terminal DNA transferase assay; TdT assay) or DNA polymerase (nick translation assay; NT assay). The biotinylated dUTP is incorporated into the chromosome at the places where abnormal DNA breaks are repaired, and are detected with fluorescein conjugated to avidin under fluorescence microscopy.

Morphology includes microscopic observations for features such as nuclear shape, and texture and contrast of nuclear and cytoplasmic staining If desired, the Pathological and Analytic Imaging Standards can be used. This database provides query capabilities for the recall and analysis of the hundreds of millions of cells represented in each dataset. Any accepted morphological criteria for evaluating glioblastoma morphology can be used.

The present disclosure is not limited in its application to the details of construction and the arrangement of components set forth in the following description. The invention described in the present disclosure is capable of other embodiments and of being practiced or of being carried out in various ways. Also, the phraseology and terminology used herein is for the purpose of description and should not be regarded as limiting.

The use of “including,” “comprising,” “having,” “containing,” “involving,” and variations thereof herein, is meant to encompass the items listed thereafter and equivalents thereof as well as additional items. For the purposes of promoting an understanding of the principles of the present disclosure, reference will now be made to preferred embodiments and specific language will be used to describe the same.

The articles “a” and “an” are used herein to refer to one or to more than one (i.e. at least one) of the grammatical object of the article. By way of example, “an element” means at least one element and can include more than one element.

### Definitions

While the following terms are believed to be well understood by one of ordinary skill in the art, the following definitions are set forth to facilitate explanation of the presently disclosed subject matter. Although any methods, devices, and materials similar or equivalent to those described herein can be used in the practice or testing of the presently disclosed subject matter, representative methods, devices and materials are now described.

The term “cell line,” as used herein, refers to individual cells, harvested cells, and cultures containing the cells, so long as they are derived from cells of the cell line referred to. A cell line is said to be “continuous,” “immortal,” or “stable” if the line remains viable over a prolonged time, typically at least about six (6) months. Preferably, the cells remain viable for at least 40 passages.

As used herein, the term “gene-defective” refers to a cell(s) which lack one or two wild-type alleles or is deficient in that particular gene function. The defects may be due to abnormal expression of the gene or a mutation in the gene itself. In certain embodiments, the gene-defective cell lacks both wild-type gene alleles, i.e., is homozygous. In other embodiments, the gene-defective cell lacks one wild-type gene alleles, i.e., is heterozygous. In some embodiments, the gene is isocitrate dehydrogenase (IDH). In certain embodiments, the gene is isocitrate dehydrogenase 1 (IDH1).

A cell line is said to be “malignant” or “tumorigenic” if, when the cell line is injected into a host animal develops tumors or cancers that are anaplastic, invasive, and/or metastatic. A “human” tumor is comprised of cells that have human chromosomes. Such tumors include those in a human patient, and tumors resulting from the introduction of a human malignant cell line into a non-human host animal for at least about one month.

The present disclosure is not limited in its application to the details of construction and the arrangement of components set forth in the following description. The invention described in the present disclosure is capable of other embodiments and of being practiced or of being carried out in various ways. Also, the phraseology and terminology used herein is for the purpose of description and should not be regarded as limiting.

The use of “including,” “comprising,” “having,” “containing,” “involving,” and variations thereof herein, is meant to encompass the items listed thereafter and equivalents thereof as well as additional items. For the purposes of promoting an understanding of the principles of the present disclosure, reference will now be made to preferred embodiments and specific language will be used to describe the same.

The articles “a” and “an” are used herein to refer to one or to more than one (i.e. at least one) of the grammatical object of the article. By way of example, “an element” means at least one element and can include more than one element.

While the following terms are believed to be well understood by one of ordinary skill in the art, the following definitions are set forth to facilitate explanation of the presently disclosed subject matter. Although any methods, devices, and materials similar or equivalent to those described herein can be used in the practice or testing of the presently disclosed subject matter, representative methods, devices and materials are described.

The term “cell line,” as used herein, refers to individual cells, harvested cells, and cultures containing the cells, so long as they are derived from cells of the cell line referred to. A cell line is said to be “continuous,” “immortal,” or “stable” if the line remains viable over a prolonged time, typically at least about six (6) months. Preferably, the cells remain viable for at least 40 passages. Such cell lines are said to grow indefinitely in culture.

A cell line is said to be “malignant” or “tumorigenic” if, when the cell line is injected into a host animal, the animal develops tumors or cancers that are anaplastic, invasive, and/or metastatic. A “human” tumor is comprised of cells that have human chromosomes. Such tumors include those in a human patient, and tumors resulting from the introduction of a human malignant cell line into a non-human host animal for at least about one week, one month, or several months.

The following examples are provided for exemplification purposes only and are not intended to limit the scope of the disclosure which has been described in broad terms above.

### EXAMPLES

### Example 1

### Generation of IDH1R132H Homozygous Human GBM Xenografts and Cell Lines

TB08-0174 (IDH1R132H homozygous) human GBM xenograft and cell lines were derived from a 31 year old female patient with a secondary human GBM. Subcutaneous tumor xenografts were generated from the tumor tissues. It has become a well-established line with over 24 passages. Genomic DNA from passage #9 xenograft has been sequenced for common glioma gene mutations. The xenograft tumor contains homozygous IDH1R132H mutation, PTEN c.333G>A, p.Q97L mutation, ERBB2 c.2444G>AG, p.G815E mutation. No mutation was identified in TP53, RB1 or PIK3R1 (X7-14). IDH1R132 mutation was also found in the primary tumor biopsy. But PTEN or ERBB2 mutations were not revealed in the tumor biopsy. The human tumor cells have been purified from the xenografts and grown in vitro.

### Example 2

### Generation of IDH1R132H Heterozygous Human GBM Xenografts and Cell Lines

TB08-0537 (IDH1R132H heterozygous) human GBM xenograft and cell lines were derived from a 37 year old female patient with secondary human GBM. Subcutaneous tumor xenografts were generated from the tumor tissues. It has become a well-established line with over 23 passages. Genomic DNA from passage #5 xenograft has been sequenced for common glioma gene mutations. The xenograft tumor contains a heterozygous IDH1R132H mutation, PTEN c.290A>T, p.Q97L mutation, and c.818G>A, p.R273H TP53 mutation. No mutation was identified in PIK3R1 (x7-14), ERBB2 or RB1. The IDH1R132, PTEN and TP53 mutations were also found in the primary tumor biopsy. The human tumor cells have been purified from the xenografts and grown in vitro.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

We claim:

1. An isolated human GBM xenograft identified as TB08-0714 (IDH1R132H homozygous).

2. An isolated human GBM cell line identified as TB08-0714 (IDH1R132H homozygous).

3. An isolated human GBM xenograft identified as TB08-0537 (IDH1R132H heterozygous).

4. An isolated human GBM cell line identified as TB08-0537 (IDH1R132H heterozygous).

5. A method for determining the effect of a test compound on DNA accumulation in an IDH1 gene-defective cell line or xenograft according to any one of the preceding claims, said method comprising:
incubating said IDH1 gene-defective cell line or xenograft in the presence and absence of the test compound;
determining and comparing DNA content in said IDH1 gene-defective cell or xenograft when incubated in the presence and absence of the test compound; and
selecting the test compound which causes DNA accumulation in said IDH1 gene-defective cell or xenograft.

6. The method of claim 5 wherein the method is performed on a pair of isogenic cell lines or xenografts.

7. A method of screening candidate agents for potential as anti-tumor agents in an IDH1 gene-defective cell line or xenograft comprising:
incubating an IDH1 gene-defective cell or xenograft according to any of claims 1-4 in the presence and absence of a candidate agent;
determining and comparing cell viability or apoptosis in the IDH1 gene-defective cell or xenograft in the presence and absence of the candidate agent; and
selecting the candidate agent as a potential anti-tumor agent which reduces cell viability or induces apoptosis in the IDH1 gene-defective cell or xenograft.

8. The method of claim 7 wherein the method is performed on a pair of isogenic cell lines or xenografts.

9. A method for screening the effect of a test compound comprising:
incubating a xenograft according to claim 1 or 3 in the presence and in the absence of the test compound;
determining and comparing morphology of the xenograft in the presence and absence of the test compound; and
selecting the test compound which reduces the tumor morphology or increases the normal morphology.

10. The method of claim 9 wherein the method is performed on a pair of isogenic cell lines or xenografts.

11. The method of claim 9 wherein the step of incubating is performed in vivo in an experimental mammal.

12. The method of claim 5 wherein the IDH1 gene-defective cell line is incubated.

13. The method of claim 7 wherein the IDH1 gene-defective cell line is incubated.

14. The method of claim 5 wherein the IDH1 gene-defective xenograft is incubated.

15. The method of claim 7 wherein the IDH1 gene-defective xenograft is incubated.
